(19) United States
(12) Patent Application Publication
(10) Pub. No.: US 2003/0187505 A1
(43) Pub. Date: Oct. 2, 2003

(54) Accommodating intraocular lens with textured haptics

(76) Inventor: Xiugao Liao, Irvine, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/113,119

(22) Filed: Mar. 29, 2002

(51) Int. Cl. A61F 2/16

(52) U.S. Cl. 623/6.37; 623/6.5

(57) ABSTRACT

An accommodating intraocular lens for implantation within the capsular bag of a human eye from which the natural lens matrix has been removed. The intraocular lens includes an optic portion for providing vision corrective power and a pair of haptics having relatively flexible portions and relatively inflexible portions reinforced with a mesh-like material such that additional stiffness is imparted to the haptic material without increasing the thickness of the haptic or changing its shape.

### FIELD OF THE INVENTION

The present invention is directed to intraocular lenses. More particularly, the invention relates to intraocular lenses which are adapted to provide bidirectional accommodating movement in the eye.

### BACKGROUND OF THE INVENTION

The physiology of the human eye includes an anterior chamber located between the cornea, or outer surface of the clear part of the eye, and the iris, the pigmented portion of the eye that is responsive to light, and a posterior chamber, filled with vitreous humor. A crystalline lens, which includes a lens matrix contained within a capsular bag, is located behind the iris and separates the iris from the posterior chamber. The crystalline lens is attached to the ciliary muscle by cord-like structures called zonules. Lining the rear of the posterior chamber is the retina, the light sensing organ of the eye, that is an extension of the optic nerve.

In young, healthy eyes, the human eye has a natural accommodation ability resulting from the contraction and relaxation of the ciliary muscle. The contraction and relaxation of the ciliary muscle acts upon the crystalline lens to provide the eye with near and distance vision, respectively. The contraction and relaxation of the ciliary muscle shapes the natural crystalline lens to the appropriate optical configuration for focusing light rays entering the eye on the retina.

As the natural crystalline lens ages, however, the structure of the lens matrix of the crystalline lens changes, becoming hazy and relatively inflexible. Eventually, the hazing of the lens matrix may progress to the point where the lens is considered cataractous, which may seriously occlude the amount of light passing through the crystalline lens and ultimately onto the retina. Fortunately, modem surgical techniques have been developed which allow removal of the cataractous lens matrix so that light may once again pass unimpeded onto the retina. However, removal of the cataractous lens matrix results in an eye that can no longer naturally accommodate to provide both near and distance vision. Even where the cataractous crystalline lens is replaced by a conventional monofocal intraocular lens, this accommodation is not recovered. Typically, one whose crystalline lens has been replaced with a conventional monofocal intraocular lens may require corrective spectacles at either distance, near, or both to provide adequate vision.

Recently, multifocal intraocular lenses have been developed to provide a person implanted with such a lens with vision at both distance near and sometimes the midrange. These lenses have multifocal optics which image both near and distance objects on the retina of the eye simultaneously. The brain then selects the appropriate image and suppresses the other image, so that a bifocal intraocular lens provides both near vision and distance vision sight without eyeglasses. Multifocal intraocular lenses, however, suffer from the disadvantage that each multifocal image focused onto the retina represents only up to 40 percent of the available light entering the eye through the cornea; the remaining light that is not focused by the multifocal optics is lost within the eye and scattered. This scattered light may result in reduction in visual acuity and/or contrast sensitivity of the eye, which may be particularly important when the wearer of such a lens is attempting visual tasks in a low-light environment, such as when trying to operate or navigate a vehicle at night.

Presently, a cataractous crystalline lens matrix is removed from an eye using a procedure whereby the cataractous natural lens matrix is extracted from the capsular bag of lens through an anterior capsulotomy, leaving the now empty capsular bag in place and attached still attached to the ciliary muscle through the zonules. Typically, the cataractous lens matrix is removed from the capsular bag through the anterior capsulotomy using phaco-emulsification and aspiration. Alternatively, the cataractous lens matrix may be removed using several other well known techniques whereby the cataractous material is broken up and aspirated from the capsular bag. After extraction of the cataractous lens matrix, an intraocular lens may be implanted within the remaining capsular bag.

Various attempts have been made to provide intraocular lenses with accommodating movement along the optical access of an eye as an alternative to take advantage of the forces applied to the capsular bag by the ciliary muscle. Typically, such lenses are biased to be located in the posterior-most position in the eye under rest or resting conditions. When near focus is desired, the ciliary muscle contracts and the lens moves forwardly providing positive accommodation. Similarly, when the visual task requires distance vision, the ciliary muscle automatically relaxes and the lens moves rearwardly to its posterior-most resting position.

Previous attempts at providing intraocular lenses that take advantage of the accommodating movement potentially provided by the ciliary muscle have utilized circular lens shapes to fully fill the capsular bag to stretch the capsular bag and maintain its shape. Other attempts have utilized plate-type designs having a central, non-flexible optic portion and relatively flexible plate-type haptics extending from the central optic to anchor the intraocular lens in the margins, or sulcus, of the capsular bag. These plate-type haptics have either been too thin to provide adequate support for the central optic and ensure that upon relaxation of the ciliary muscle that the lens returns to its posterior position, or the plate-type haptics have been made thick enough to provide stability, which results in the haptic being relatively inflexible, requiring the addition of a hinge-like structure extending across the width of the plate haptic to ensure adequate flexibility to allow for lens motion in the eye to provide accommodation.

What has been needed and heretofore unavailable, is an accommodating intraocular lens having haptics incorporating varying zones of flexibility. The varying zones of the flexible haptics would include areas of the haptic which are relatively flexible to allow the haptic to bend in response to forces applied on the lens by the ciliary muscle to provide the accommodating motion necessary for an accommodating intraocular lens. The varying zones would also include zones or areas where the haptic has been reinforced or stiffened to make the haptic relative inflexible compared to the flexible areas or zones of the haptics to assist in correctly positioning and maintaining the position of the accommodating intraocular lens in the capsular bag. Moreover, the haptics of such an accommodating lens should provide for in growth of fibrotic material to ensure firm fixation of the accommodating lens is firmly fixed in position in the capsular bag and to ensure that forces applied to the capsular bag by the ciliary muscle will be efficiently transmitted to the accommodating lens without unwanted movement of the haptics of the lens within the capsular bag during the contraction and relaxation of the ciliary muscle during accommodation.

### SUMMARY OF THE INVENTION

The invention provides for improved designs of accommodating intraocular lenses. The accommodating intraocular lenses of the present invention have generally rectilinear plate-style haptics having varying zones or areas of flexibility so as to enable the haptics to maintain the centration and fixation of the intraocular lens in the capsular bag of an eye after extraction of the matrix of a natural lens. The varying zones of flexibility also enable the haptics to flex in accordance with constriction and relaxation of the ciliary muscle of the eye to move the optic of the intraocular lens along the visual axis of the eye to change the focus of light passing through the intraocular lens onto the retina of the eye, thus providing visual accommodation.

One embodiment of the present invention is an accommodating intraocular lens comprising an optic adapted to focus light toward a retina of an eye, and a pair of generally rectilinear plate haptics joined to and extending from opposite sides of the optic, each of the pair of generally rectilinear plate haptics having a flexible portion and a relatively inflexible portion. The relatively inflexible portion of the haptics includes a material having a flexibility less than the flexibility of the flexible portion for providing increased stiffness disposed within a thickness of the relatively inflexible portion of the haptics. The pair of generally rectilinear plate haptics have a length, a width and a thickness and the thickness of the plate haptics which may be substantially equal along the length and width of the haptics. In one embodiment, the generally rectilinear haptics are joined to the optic in such a manner so that the longitudinal axis of the haptics lie in the same plane as the optic. In another embodiment, the haptics are joined to the optic in such a manner that the longitudinal axis of the haptics do not lie in the same plane as the optic, thus the haptics are angulated with respect to the optic.

In one embodiment, the relatively inflexible portion includes a material for providing increased stiffness disposed within the thickness of the relatively inflexible portion. In another embodiment, the material for providing increased stiffness may be formed as a mesh.

In another embodiment of the present invention, each of the pair of plate haptics has a proximal end joined to the optic and a distal end and at least one of the pair of plate haptics has at least one opening extending through the plate haptic adjacent the distal end of the at least one plate haptic. In some embodiments, the opening may be located adjacent the distal end of the plate haptic.

In yet another embodiment, the surface of the haptics may be smooth and non-tacky. In an alternative embodiment, the surface of the haptics may be textured, or it may be smooth and tacky, to enhance fixation of the haptic in the fibrotic tissue that forms when the anterior and posterior capsular walls fibrose after removal of the matrix of the natural lens. In still further embodiments, the distal ends of the haptics may be formed in complex shapes, such as arms or foot-like tabs to enhance fixation. In still further embodiments, openings or cut-outs may be formed in or adjacent to the distal end of the haptics to enhance growth of fibrotic tissue around or through the haptic to fixate the lens.

In still another embodiment of the accommodating intraocular lens of the present invention, each of the pair of plate haptics has a proximal end joined to the optic and a distal end and further comprising a fixation element having a distal portion and a proximal end attached to the distal end of at least one of the pair of plate haptics. The proximal end of the fixation element may be disposed within the thickness of the distal end of the plate haptic. Additionally, the fixation element may have a flexible distal portion that is capable of flexing from a first position to a second position.

In yet another embodiment, the accommodating intraocular lens of the present invention has an optic portion having a width, and the plate haptics joined to the optic portion have proximal and distal ends, the distal ends having a width that may be substantially the same as the width of the optic portion, or the width of the distal ends may be different that the width of the optic portions. In one alternative embodiment, the width of the distal end of the haptic may be greater than the width of the optic portion, which in another embodiment, the width of the distal end of the haptic may be less than the width of the optic portion.

In still another embodiment, the present invention includes designs for accommodating intraocular lens having an optic portion and haptics having flexible and relatively inflexible portions where the relatively inflexible portions incorporate a material having less flexibility that the flexible portion to provide stiffness to the relatively inflexible portion. The material disposed within the relatively inflexible portion may have a solid structure in one embodiment, or a mesh-like structure in another embodiment.

Other features and advantages of the present invention will become more apparent from the following detailed description, taken in conjunction with the accompanying drawings, which illustrate, by way of example, the principles of the invention.

### DESCRIPTION OF THE DRAWINGS

FIG. 1 is a cross-sectional view in which an accommodating intraocular lens of the present invention is implanted, the lens being located in a posterior position in the eye;

FIG. 2 is a cross-sectional view of an eye in which the accommodating intraocular lens of the present invention is shown with the lens being located in an anterior position in the eye;

FIG. 3A is a top view of an embodiment of the present invention having a central optic and a pair of plate-type haptics having flexible and reinforced relatively inflexible zones;

FIG. 3B is a side view of the embodiment of FIG. 3A showing angulation of the haptics;

FIG. 3C is a cross-sectional view of the embodiment of FIG. 3A;

FIG. 4A is a top view of another embodiment of the present invention having a central optic and a pair of slightly convex plate-type haptics having flexible and relatively inflexible zones, the distal end of the haptics also having slots for providing space for fibrosis to extend through the lens to anchor the lens in place;

FIG. 4B is a side view of the embodiment of FIG. 4A;

FIG. 5A is a top view of yet another embodiment of the present invention having a central optic and a pair of plate-type haptics having distal ends formed in a complex shape to provide anchoring of the lens in the capsular bag by allowing fibrosis to surround portions of the intraocular lens;

FIG. 5B is a side view of the embodiment of FIG. 5A;

FIG. 6A is a top view of another embodiment of the present invention having a central optic and a pair of plate-type haptics having flexible and relatively inflexible zones with the distal end of each haptic having a pair of arms, each arm including a hole extending through the haptic to provide for anchoring of the lens in the capsular bag;

FIG. 6B is a side view of the embodiment of FIG. 6A;

FIG. 7A is a top view of still another embodiment of the present invention having a central optic and a pair of plate-type haptics having flexible and relatively inflexible zones and including a slot located at the distal end of each haptic;

FIG. 7B is a side view of the embodiment of the present invention in 7A;

FIG. 8A is a top view of another embodiment of the present invention having a central optic and a pair of plate-type haptics having flexible and relatively inflexible zones and including a pair of slots located at the distal end of each haptic;

FIG. 8B is a side view of the embodiment of the present invention depicted in FIG. 8A;

FIG. 9A is a top view of yet another embodiment of the present invention having a central optic and a pair of plate-type haptics having flexible and relatively inflexible zones and wherein the distal end of each haptic is formed to provide a pair of tabs, each tab having a hole extending through the haptic;

FIG. 9B is a side view of the embodiment of the present invention shown in FIG. 9A;

FIG. 10A is a top view of another embodiment of the present invention having a central optic and a pair of plate-type haptic portions having flexible and relatively inflexible zones and incorporating a T-shaped fixation element anchored within each plate-type haptic adjacent the distal end of the haptic;

FIG. 10B is a side view of the embodiment of the present invention depicted in FIG. 10A;

FIG. 11A is a top view of an another embodiment of the present invention having a central optic and a pair of plate-type haptics having flexible and relatively inflexible zones, each plate-type haptic formed to have a pair of tabs located at the distal end of the haptic, and including a loop-style haptic having a proximal end anchored in one of the tabs of each haptic diagonally across from the loop mounted on the other side of the optic, each loop also having a hole located adjacent a distal end of the loop; and

FIG. 11B is a side view of the embodiment of the present invention shown in FIG. 10A.

### DETAILED DESCRIPTION OF THE PREFERRED EMBODIMENTS

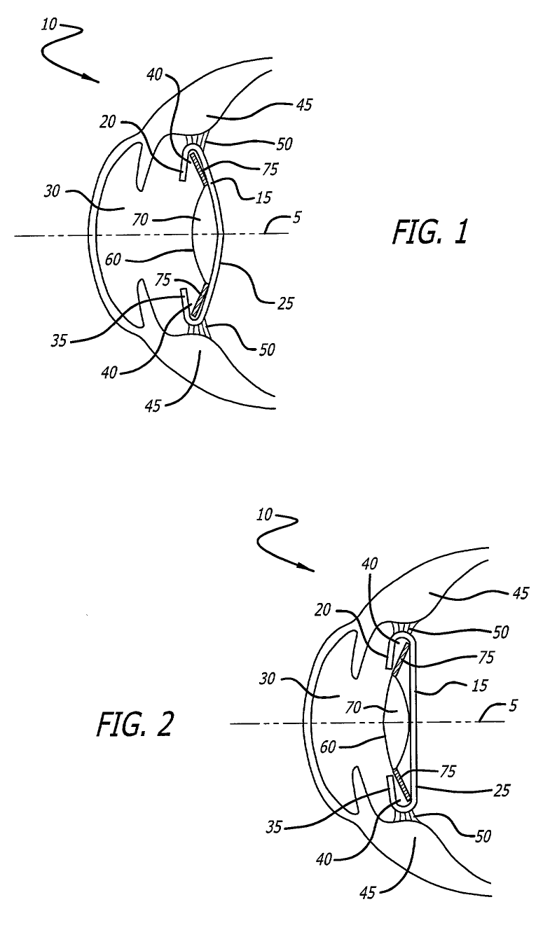

FIG. 1 depicts a human eye 10 from which the natural crystalline lens matrix was previously removed by a surgical procedure involving an anterior capsulotomy 30 in the anterior wall 20 of the capsular bag. The natural lens comprises a lens capsule, also called the capsular bag, have elastic anterior and posterior walls 20, 25 respectively, which are referred by ophthalmologists and herein as anterior and posterior capsules, respectively. Within the capsular bag 15 is a normally optically clear crystalline lens matrix (not shown). In many individuals this lens matrix becomes cloudy with advancing age and forms what is called a cataract, which may seriously obstruct light passing through the lens onto the retina so that a person with such a cataractous lens suffers a reduction in their visual acuity. It is now common practice to restore a cataract patient's vision by removing the cataract from the natural lens and replacing the cataractous lens matrix with an artificial intraocular lens.

A common surgical procedure in removing cataractous lens matrixes involves the formation of an anterior capsulotomy 30, or opening within the anterior capsular wall 20 of the cataractous lens. Once this opening is made, the cataractous lens matrix may be removed from the interior of the capsular bag 15 using either phacoemulsification or some other method to remove the cataractous lens matrix through the anterior capsulotomy 20.

Typically, great care is taken to remove as much of the lens matrix as possible and to ensure that the remaining anterior and posterior capsular surfaces are free from lens matrix material. As shown in FIG. 1, after an anterior capsulotomy 30 and lens matrix removal, the capsular bag 15 includes an annular anterior capsular wall remnant or rim 35 and an elastic posterior capsular wall 25 which are joined along the perimeter of the bag to form an annular crevice-like capsular bag sulcus 40 between the anterior wall rim 35 and posterior capsular wall 25. The capsular bag 15 is the remnant of the of the natural lens which remains after the anterior capsulotomy has been performed on the natural lens and the cataractous lens matrix has been removed. The capsular bag 15 is secured about its perimeter to the ciliary muscle 45 of the eye by zonules 50.

Natural accommodation in a normal human eye having a normal human crystalline lens involves automatic contraction or constriction and relaxation of the ciliary muscle 45 of the eye by the brain in response to looking at objects at different distances. Ciliary muscle relaxation, which is the normal state of the muscle, as shown in FIG. 1, shapes the human crystalline lens for distance vision. Ciliary muscle contraction, as shown in FIG. 2, shapes the human crystalline lens for near vision. The brain-induced change from distance vision to near vision is referred to as accommodation.

Implanted within the capsular bag 15 of the eye 10 is an accommodating intraocular lens 60 according to this invention which replaces and performs the accommodation function of the removed human crystalline lens. Lens 60 as depicted in FIG. 1 is commonly referred to as a plate-type haptic lens It will be understood by those skilled in the art that the accommodating intraocular lens of the present invention may be utilized to replace either a natural lens which is virtually total defective, such as a cataractous natural lens, or a natural lens that provides satisfactory vision at one distance without the wearing of glasses, but provides satisfactory vision at another distance only when glasses are worn. For example, the accommodating intraocular lens of the present invention can be utilized to correct refractive errors and restore accommodation for persons in their mid-40's and latter who have become presbyopic and who require reading glasses, bifocals, or trifocals or other optical aids to provide near vision, but who may not have a cataract.

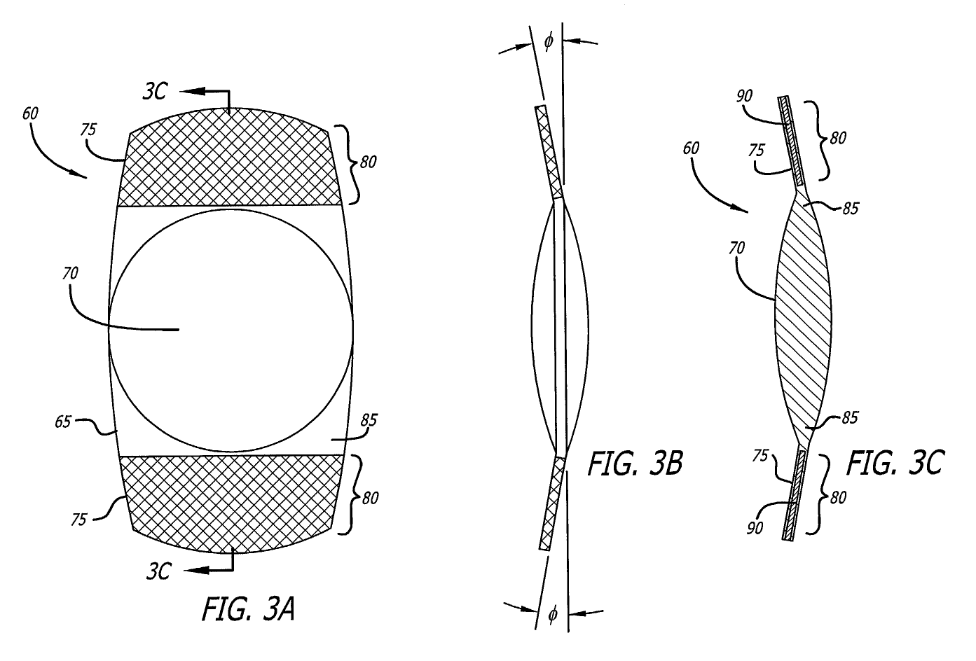

FIG. 3A illustrates the principles of the present invention depicting an intraocular lens 60 having a body 65 which may be formed of a relatively hard material, a relatively soft, flexible semi-rigid material, or a combination of both hard and soft materials. Examples of relatively hard materials which are suitable for the lens body are methylmethacrylate, polycarbonate, polysulfone, and other relatively hard biologically inert optical materials. Examples of suitable relatively soft materials for the lens are silicone, hydrogel, soft hydrophobic acrylic material, thermolabile materials, and other flexible semi-rigid biologically inert optic materials.

As seen from the illustration in FIG. 3A, the lens body 65 has a generally rectangular shape and includes a central optic zone or optic 70 and plate-type haptics 75 extending from diametrically opposite edges of the optic 70. The haptics have an inner, or proximal, end joined to the optic and opposite outer, or distal, free end. The outer, or distal, ends of haptics 75 are movable anteriorly and posteriorly relative to the optic. The particular lens embodiment illustrated may be constructed of a resilient, semi-rigid material and have haptics having varying zones of flexibility.

The haptics 75 do not have the same flexibility along the entire length of the haptic. The outer areas 80 of the haptic 75 have been reinforced to provide an adequate amount of stiffness to reduce the flexibility of the haptic 75 to support the lens in the capsular bag. The inner portions 85 of the haptic 75, however, are relatively flexible and allow flexing of the haptic in this area so that force applied to the relatively inflexible, reinforced areas 80 of the haptics 75 result in movement of the optic 70 along the visual axis of the eye 5 (FIG. 1). This deflection of the optic along the axis 5 of the eye is shown in FIG. 2, wherein contraction of the ciliary muscle applies a force to the capsular bag 15 which causes the plate-type haptics 75 of the intraocular lens 60 to flex in the flexible regions 85, causing the optic 70 of the intraocular lens 60 to move anteriorly, or towards the cornea, along the visual axis 5 of the eye.

The accommodating intraocular lens 60 of the present invention is implanted within the capsular bag 15 of the eye 10 in the position shown in FIG. 1. When implanting the lens in the bag, the ciliary muscle 45 of the eye 10 is maintained in a relaxed state, as depicted by the position of the capsular bag 15 in FIG. 1. In this relaxed state, the capsular bag is stretched to its maximum diameter. The intraocular lens 60 is inserted into the capsular bag 15 through the anterior capsulotomy 30, and placed in the position shown in FIG. 1. In this position, the lens optic 70 is aligned on the axis 5 of the eye with the anterior capsulotomy 30, and the posterior side of the lens faces, and typically is in contact with, the elastic posterior capsular wall 25 of the capsular bag 15.

As shown in FIGS. 1 and 2, the outer or distal ends of the lens haptics 75 are situated within the sulcus 40 at the radially outer perimeter of the capsular bag 15. The overall length of the intraocular lens substantially equals the inner diameter of the stretched capsular bag 15 so that the lens 60 fits snugly within the stretched capsular bag 15, as shown. Typically, the length of the intraocular lens 60 will have an overall lens diameter of between 9.0 mm to 13.5 mm, and more particularly an overall diameter of 10 mm to 12 mm. Fitting a suitably size lens into the sulcus 40 of the capsular bag 15 ensures centration of the intraocular lens 60 and permits the optic 70 to move forward inside the capsular rim 35 during accommodation. Typically, the central optic 70 of the intraocular lens 60 will have a diameter between about 3 mm and 7.5 mm and preferably an optical diameter between about 5 mm to 6.5 mm.

The width of the plate-type haptics 75 of the intraocular lens 60 may have the same width as the central optic. Alternatively, the width of the plate-type haptics 75 may taper gradually from the proximal to the distal end of the haptic 75 such that the distal end of the haptic is not as wide as the central optic. Alternatively, the width at the distal edge of the haptic 75 may be greater than the width of the optic 70. The thickness of the plate-type haptic 75 is typically from about 0.05 mm to about 0.5 mm. The plate-type haptic may angle from the optical body as depicted in FIG. 3B. Typically, the plate-type haptic 75 will have an angle in a range of about 0° to 20° relative to the optic 70, and preferably the angle between the optic 70 and the haptic 75 will be in a range between about 0° and 12°.

The surface of plate-type haptics 75 may be either smooth or frosted. Alternatively, the surface of plate-type 75 haptics may either be smooth and untacky, or the surface may be treated to form a somewhat tacky surface. Additionally, the surface of haptics 75 may be treated in such a way as to induce the growth of fibrotic tissue on the surface of haptics 75 to significantly increase the anchoring of haptics 75 within the capsular bag 15 and thus assist in maximizing the accommodation provided by the lens 60 by ensuring that forces on the capsular bag 15 caused by contraction and relaxation of the ciliary muscle 45 are efficiently coupled to the lens 60 to move the optic 70 of the lens 60 along the axis 5 of the eye. The optic or optic zone 70 of the lens 60 may be either a monofocal optic, a toric optic, or a multifocal optic. Alternative designs of the optic portion 70 of lens 60 are also possible, as should be apparent to those skilled in the art, without departing from the scope of the present invention.

As shown in FIG. 3C, the portion 80 of the haptic 75 that is reinforced may be reinforced by including a mesh 90 of reinforcing material within the thickness of the haptic 75. This mesh 90 of material provides for reinforcement of the relatively thin haptic, thus rendering the reinforced portion 80 of haptic 75 substantially less flexible relative to the flexible portion 85 of haptic 75. This allows the use of relatively thin and flexible haptics 75 to provide accommodation in the flexible areas 85 surrounding the optic 70, but reinforces the peripheral, or distal, portions 80 of the haptic 75 without increasing the thickness of the haptic 75. Such reinforcement also eliminates the need to join or otherwise graft another, less rigid, material to the haptic 75 to decrease the flexibility of the haptic. The mesh 90 used to reinforce the plate-type haptics 75 of the present invention may be a plastic material such as polypropylene, polyethylene, copoly(propylene and ethylene), copoly(propylene and butylene) polyvinylidine fluoride (sold by ATOFINA Chemicals, Inc. under the registered tradename KYNAR) and the like. The mesh may be incorporated into the peripheral portion 80 of lens 60 during molding of the lens. Alternatively, a solid, non-mesh, form of the materials described above may be used to reinforce and stiffen the haptics.

The lens 60 may be formed using a molding process suitable for the material used to form the lens. For example, lens 60 may be injection molded or compression molded. Alternatively, a lens blank or rough may be formed having incorporated therein a central section from which the optic 70 will be formed and a peripheral portion reinforced with mesh 90 from which the haptics 75 will be formed. The final lens could be produced from the blank using precision lathe cutting and polishing to form the desired surfaces of the optic 70 and haptics 75. Typical materials for forming the lens 60 have been described previously.

During a post-operative healing period on the order of two to three weeks following surgical implantation of the accommodating intraocular lens 60 in the capsular bag 15, epithelial cells under the anterior capsular rim 35 of the capsular bag 15 proliferate and typically cause the anterior rim 35 to fuse to posterior capsular wall 25 by a process known as fibrosis. Because the haptic 75 of lens 60 extends into the sulcus 40 of the capsular bag, the haptic 75 is generally surrounded or encapsulated by the fibrosis, anchoring the haptics 75 into position in the capsular bag 15. Where there are no holes or other apertures extending through the haptic 75, the haptic resides in a pocket formed within the fibrosis. Where the haptic 75 has been formed having a complex shape, or includes holes or other apertures, the fibrosis may extend through the haptics 75 to further anchor the haptics 75 within the pocket of fibrotic material.

In order to ensure proper formation of the haptic pockets, sufficient time must be allowed for fibrosis to occur to completion without flexing of the lens haptics by ciliary muscle action. One way of accomplishing this is to have the patient periodically administer cycloplegic drops, such as atropine, into the patient's eye during the post-operative fibrosis period. These drops maintain the ciliary muscle 45 in its relaxed state and prevent premature contraction of the ciliary muscle 45 which might cause one or both of the haptics 75 to be dislodged from their respective fibrotic pockets, which could lead to less than satisfactory performance of the accommodating function of the lens. In the worst case, the surgeon may have to re-enter the eye to manipulate and/or reposition the haptics 75 of lens 60 back into the sulcus 40.

The anterior capsular rim 35 shrinks during fibrosis and thereby shrinks the capsular bag 15 slightly in its radial direction. This shrinkage combines with the anchoring of the lens haptics 75 to produce opposing end wise compression forces on the ends of the haptics 75 which tend to buckle or flex the lens in the flexible portion 85 of the haptics 75, thereby causing the optic 70 of the lens 60 to move along the axis 5 of the eye.

The accommodating intraocular lens 60 of the present invention is uniquely constructed to utilize the same ciliary muscle action that shapes the natural lens to focus the eye at different distances to effect accommodative movement of the lens optic 70 along the optic axis 5 of the eye between the distance vision position as shown in FIG. 1 to the near vision position as shown in FIG. 2. Thus, when looking at a distance scene, the brain relaxes the ciliary muscles 45. Relaxation of the ciliary muscles stretches the capsular bag 15 to its maximum diameter and causes its fibrous anterior capsular rim 35 to become taught. The taught rim 35 deflects the lens optic 70 rearwardly to its posterior distant vision position. When looking at a near scene, such as a book when reading, the brain constricts or contracts the ciliary muscle 45 as shown in FIG. 2. This ciliary muscle contraction has the three-fold effect of increasing the vitreous cavity pressure, relaxing the capsular bag 15 and in particular its fibrosed anterior capsular rim 35, and exerting opposite end wise compression forces on the ends of the lens haptic 75 which results in end wise compression of the lens 60. Relaxation of the anterior capsular rim 35 permits the rim to flex forwardly and thereby enable the combined forward bias force exerted on the lens 60 by the rearwardly stretched posterior capsule and increased vitreous cavity pressure to push the lens forwardly in an initial accommodative movement from the position of FIG. 1 to the position of FIG. 2.

The lens haptics 75 flex in their flexible portions 85 with respect to the lens optic 70 during accommodation. Any elastic strain energy forces developed in the flexible portion 85 during this flexing produces additional anterior and/or posterior forces on the lens 60. The lens 60 may be designed to assume any normal unstressed position to either aid or resist accommodation of the lens in a near position and assist in returning the lens 60 to its distance position depending on the unstressed position of the lens.

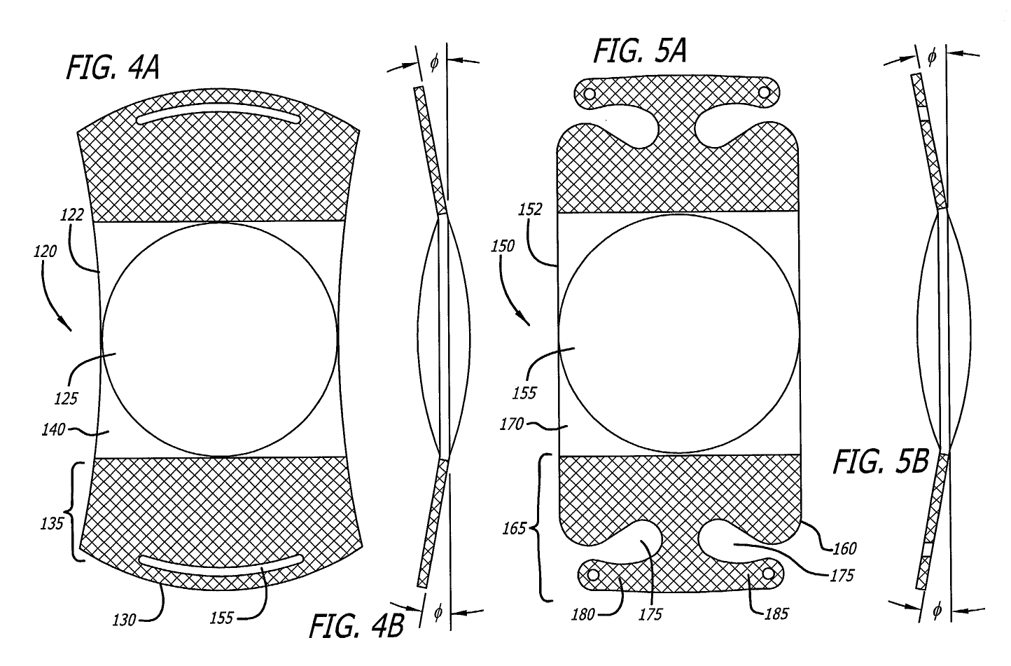

FIGS. 4A to 11B illustrate modifications to the accommodating intraocular lenses of the present invention. As will be obvious to those skilled in the art, the illustrated embodiments are not exhaustive and are not limiting, but are merely examples of the various accommodating intraocular lens designs incorporating aspects of the present invention that are possible. These additional embodiments incorporate various means for fixating or anchoring the lens haptics in the capsular bag 15 (FIG. 1) to prevent the lenses from entering the posterior chamber of the eye in the event that the posterior capsular wall 25 becomes torn or when a posterior capsulotomy must be performed on the posterior capsular wall 25 to create an aperture in a capsular wall 25 that has become hazy or opaque due to fibrosis. It will be understood by those skilled in art that the additional embodiments shown in FIGS. 4A to 11B are simply variations of the lens design embodying the inventions of previously described in reference to FIGS. 3A, 3B and 3C and are implanted in the capsular bag 15 of the eye 10 (FIG. 1) in the same manner as described in connection with FIGS. 3A-3C.

The accommodating intraocular lens depicted in FIGS. 4A and 4B is similar to the accommodating intraocular lens described with reference to FIGS. 3A-3C and has a lens body 122 having an optic 125 and a pair of haptics 130 attached thereto. Haptics 130 have an outer reinforced portion 135 and an inner, flexible, portion 140. The reinforced portion 135 of haptics 130 is formed by incorporating a relatively inflexible material, such as the mesh or solid material described above in reference to FIGS. 3A-3C. Situated adjacent the peripheral edges of the reinforced portion 135 of haptics 130 are slots 150. Slots 155 allow for fibrosis of the anterior capsular rim 35 to anchor the lens 120 in position in the capsular bag 15 by providing a path for fibrosis to form between the anterior capsular rim 35 and the posterior capsular surface wall 25. Lens 120 is thus firmly fixated in position within the capsular bag capsular bag 15, ensuring that when force is applied to the rim of the capsularbag 15 by ciliary muscles 45 through the zonules 50, any motion imparted to the bag by the ciliary muscle 45 is transmitted to the body 122 of lens 120 without dislodging the peripheral end of one or both haptics 130.

Another embodiment of the present invention is depicted in FIGS. 5A and 5B. In this embodiment, an accommodating intraocular lens 150 having a body 152, an optic 155 and haptics 160 is shown. As in previous embodiments, haptics 160 includes a reinforced section 165 and flexible section 175. The reinforced section 165 is formed by incorporating a relatively inflexible material, such as is described above. Additionally, the peripheral portion of haptic 160 is formed having a distal T-shaped area by removing material during manufacturing from the peripheral portion 165 of haptic 160. The resulting shape of the peripheral portion 165 incorporates one or more complex curves that form cut-ins in the peripheral portion 165, resulting in the distal end 180 of the peripheral portion 165 of the haptic 160 having a substantially T-shape. Alternatively, instead of removing material to form the cut-ins 175, the cut-ins 175 may be formed by molding the lens 150 into the desired shape. In yet another embodiment, the T-shaped distal portion 180 of haptic 160 may include one of more holes 185 formed distal to the cut-ins 175. While holes 175 are shown in FIG. 5A located adjacent the ends of the head of the substantially T-shape, it will be understood by those skilled in the art that holes 175 may be formed anywhere along the top of the T-shaped portion 165 without departing from the scope of the present invention. Additionally, while a T-shape distal end of the haptics 160 is shown, it will be understood that other designs are contemplated, depending only on the design requirements of the accommodating intraocular lens, without departing from the scope of the present invention.

As described previously, forming cut-ins 175 and/or holes 180 provides for improved anchoring of the haptics 160 of lens 150 during formation of the fibrosis of the capsular bag 15. The T-shaped distal portion 180 of haptics 160 allows for ingrowth and subsequent fibrosis of endothelial cells through cut-ins 175, thus anchoring the T-shaped distal portion 180 of haptics 160 firmly within the fibrosed capsular bag. Additionally, holes 185 may be sized to accommodate a suture thread so that, in those instances where the capsular bag may be ripped, or otherwise incapable of supporting the lens, a suture may be placed between the tip of the haptic and a portion of the eye to hold the lens in place.

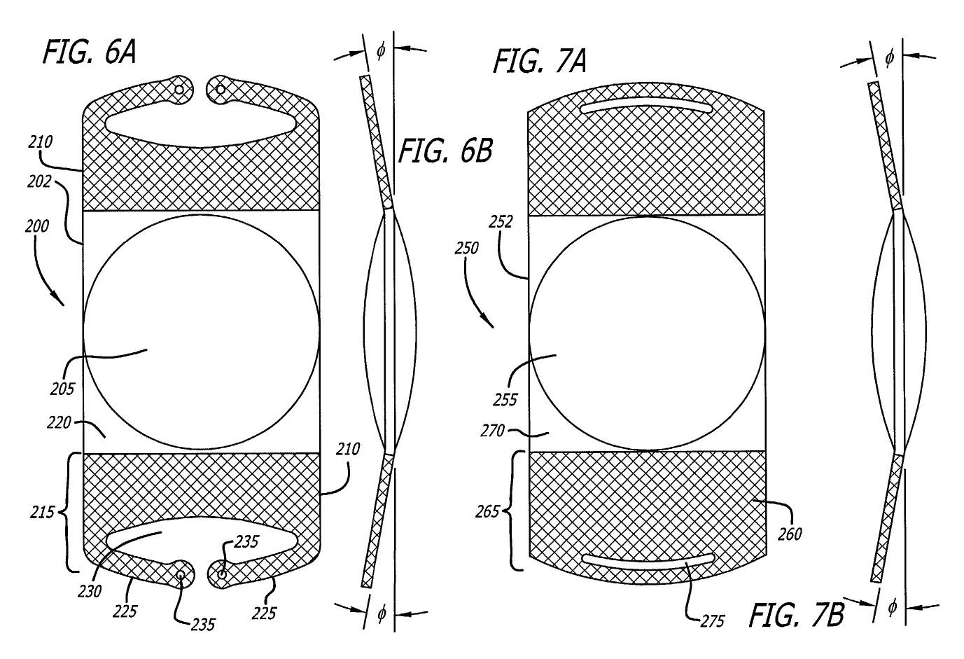

FIGS. 6A and 6B depict another embodiment of an accommodating intraocular lens according to the present invention. In this embodiment, accommodating intraocular lens 200 includes a body 202 having an optic portion 205 and a pair of haptics 210. Haptics 210 include a reinforced portion 215 and a non-reinforced flexible portion 220. The reinforced portion 215 may be formed by incorporating a relatively inflexible material, such as the mesh described above, into the haptics in an area selected to provide the desired amount of stiffening.

The reinforced portion 215 of haptic 210 includes a pair of arms 225 integrally formed adjacent the distal ends of each of the haptics 225. Alternatively, lens 200 may be formed so that only one the pair of haptics 210 includes a pair of arms 225. Thus, in accordance with the embodiment depicted in FIG. 6A, accommodating intraocular lens 200 may have a total of 4 arms 225, one pair situated at the peripheral, or distal, ends of each of haptics 225. Alternatively, lens 200 may be formed so that only one the pair of haptics 210 includes a pair of arms 225.

As depicted in FIG. 6A, reinforced portion 215 of haptic 225 includes a cut-out are 230, forming arms 225. Depending on the width of the arms 225, the arms may be somewhat flexible or relatively inflexible. For example, arms 225 may be formed having a width substantially thin enough so that arms 225 may flex slightly as the capsular bag contracts during fibrosis. Additionally, this flexure of arms 225 may also assist in centering the lens 200 within the capsular bag 15 during implantation of the lens which allows the spring arms 225 to deflect towards the optic portion 205 of intraocular lens 200. For example, an embodiment of the accommodating intraocular lens 200 having flexible arms 225 is advantageous in that the flexibility of arms 225 allows the intraocular lens 200 to be implanted within a capsular bag 15 (FIG. 1) that may be slightly smaller in diameter than optimal for the implantation of intraocular lens 200, or which may have some non uniformity in the shape of the sulcus 40 of the capsular bag 15. The spring-like nature of the integrally formed arm 225 allows the arms to compress slightly when the lens is implanted. Additionally, the spring like nature of arms 225 also allows for compression of arms 225 towards the optic portion 205 during fibrosis of the lens capsule after the lens is implanted. Moreover, cut-out 230 allows the anterior and posterior portions of the lens capsule to fibrose together, forming pockets within the fibrosis that capture arms 225, thus ensuring adequate anchorage and fixation of intraocular lens 200 in the capsular bag 15.

In an additional embodiment, one or more holes 235 may be formed in one or more ends of arms 225. While FIG. 6A depicts holes 235 as being formed in the end of arms 225 furthest from where arms 225 connect with the remainder of the reinforced portion 215 of haptic 225, it will be understood that holes 235 may formed at any location along arms 225. Similar to the holes formed in the haptics of previously described embodiments, holes 235 allow for additional anchoring and fixation of the intraocular lens in that they allow fibrosis through the hole or, alternatively, to allow fixation of the lens during implantation using a suture threaded through the hole to attach lens 200 to a portion of the eye. Alternatively, and without limitation to the embodiment depicted in FIG. 6A, a suture may be threaded through the holes 235 in such a way as to hold the lens 200 in a folded or compressed state during implantation of the lens 200 through a small incision in the eye and through a relatively small anterior capsulotomy 30 (FIG. 1). Once lens 200 is inserted into the capsular bag 15, the suture may be pulled through the holes 235, releasing the arms 235 and haptics 210, allowing the haptics 210 to take on a normal, non-folded or non-compressed shape.

FIGS. 7A and 7B depict an additional embodiment of the present invention similar to the embodiment described above with reference to FIGS. 3A-3C. This embodiment is similar to that depicted in FIGS. 4A and 4B, and the description with reference to those figures applies equally to FIGS. 7A and 7B. The accommodating lens 250 of this embodiment has a lens body 252 having an optic 255 and a pair of haptics 260 attached thereto. Haptics 260 have an outer reinforced portion 265 and an inner, flexible, portion 270. The reinforced portion 265 of haptics 260 is formed by incorporating a relatively inflexible material, such as the mesh described above. Situated adjacent the peripheral, or distal, edges of the reinforced portion 265 of haptics 260 are slots 275. As should be apparent when comparing the embodiments illustrated in FIGS. 4A and 7A, lens body 122 (FIG. 4A) is formed so that the width of the distal end of haptics 130 of lens 120 is wider than the width of the optic 125 of lens 120 while the width of the distal end of haptics 260 of lens 250 (FIG. 7A) is substantially the same as the width of the optic 255 of lens 250.

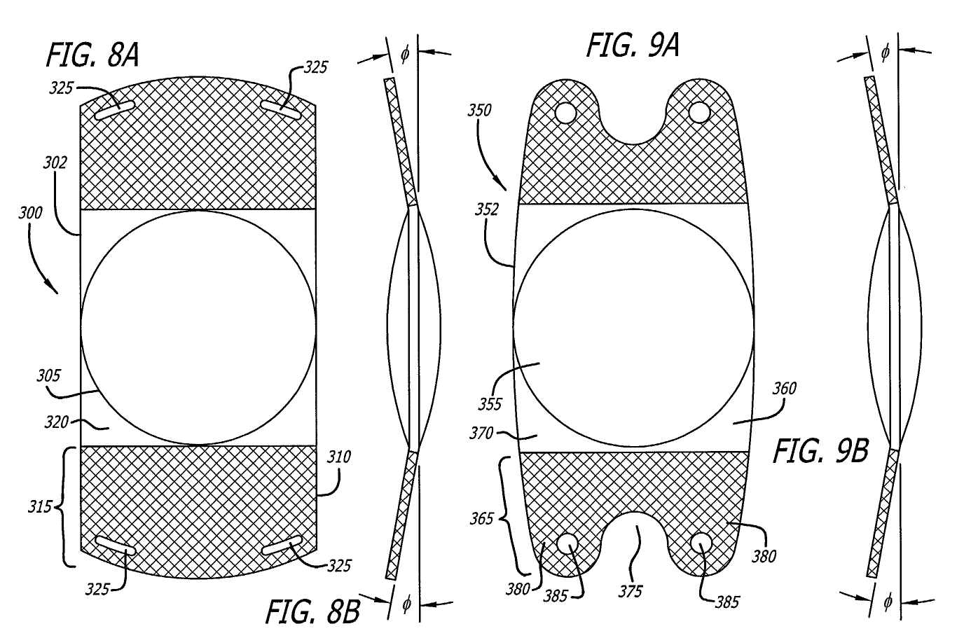

Similarly, the embodiment depicted in FIGS. 8A and 8B is similar to the embodiment of FIG. 7A, except that, rather than having a single slot located in the medial portion of the distal end of haptics 260 of lens 250 as shown in FIG. 7A, the embodiment depicted in FIG. 8A has a pair of slots 325 formed in haptics 310 of lens 300 adjacent the peripheral corners of the distal end of the reinforced portion 315 of haptics 310 of lens 300. As will be understood by those skilled in the art, the number of slots 325 is not limited to the depicted embodiment. More or less slots 325 may be formed in haptics 310 as desired without departing from the scope of the present invention.

A further embodiment of the present invention is illustrated in FIGS. 9A and 9B. In this embodiment, an accommodating intraocular lens 350 is shown having a body 352 including an optic portion 355 and a pair of haptics 360. The haptics 360 include a reinforced portion 365 and a non-reinforced, relatively flexible portion 370. The reinforced portion 365 of haptics 360 are formed by incorporated a relatively inflexible material, such as the mesh described above, into the haptic 360. As shown in the embodiment depicted in FIG. 9A, the distal end of haptics 360 may have a width that is less than the width of the optic portion 355 of lens 350. Alternatively, the width of the distal ends of haptics 360 may be greater than, or equal to, the width of the optic portion 355.

Accommodating intraocular lens 350 may also include a cut-out 375 formed in one or both of haptics 360 to provide a pair of tabs or foot-like shapes 380 to assist in locating and fixating lens 350 in the capsular bag 15. Cut-out 375 may be formed by removing a portion of haptic 360, or, alternatively, cut-out 375 may be formed by molding the lens using molding techniques well known to those skilled in the art. As in previous embodiments, one or more holes may be formed in tabs 380 to provide for growth of fibrotic tissue through the haptic 360. While FIG. 9A shows lens 350 having holes 385 formed in each tab 380, it is not necessary to form holes in each tab. For example, in one embodiment, holes 385 may formed in the tabs of haptics 360 such that holes are formed only in the tabs 380 located diagonally across optic portion 355 from one another.

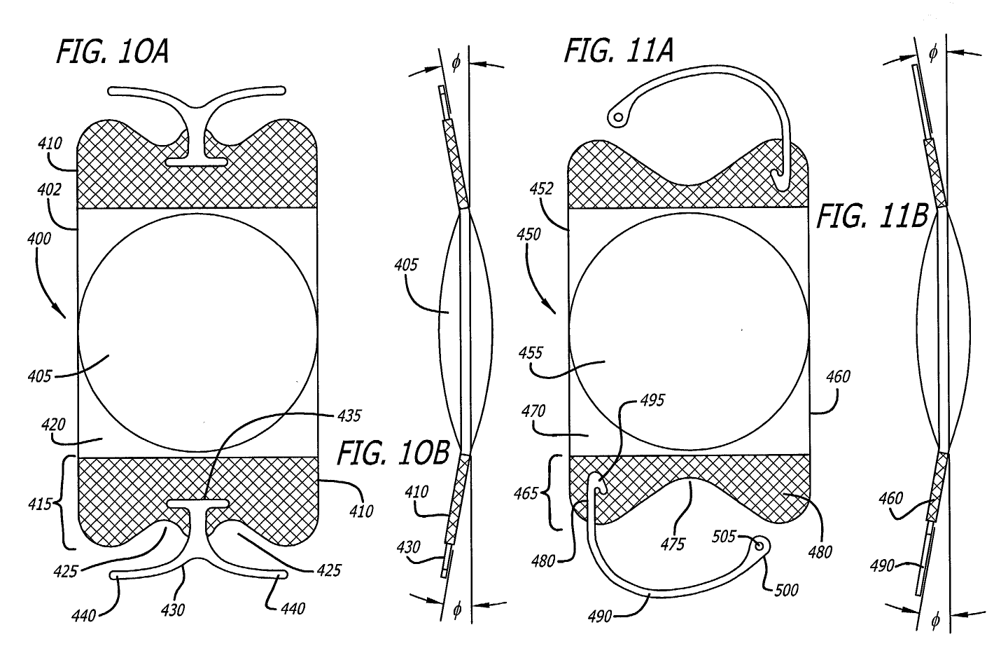

The accommodating plate-type haptic lenses described herein above are intended for use when the anterior capsulotomy and subsequent removal of the lens matrix results in an intact capsular remnant or rim that is circumferentially continuous and which has a width sufficient to capture the peripheral edge of the plate-type haptic to retain the lens in the proper position within the capsular bag during and/or after fibrosis, although such designs may be used in other situations as determined to be appropriate by a physician. The present invention, however, is not limited to simple plate-type haptics, and can be modified as shown in FIGS. 10A, 10B, 11A and 11B for use when the anterior capsule or remnant or rim of the capsular bag is captured, cut, or torn, or has too small a radial width to firmly retain the lens in proper position during and/or after fibrosis.

A ruptured anterior capsule or remnant or rim, or one which does not have sufficient radial width, may preclude utilization of a simple plate-type haptic lens for the following reasons. A ruptured rim may not firmly retain the lens haptics in the sulcus of the capsular bag during fibrosis. This renders the lens prone to decentration and/or dislocation, such as dislocation into the vitreous cavity if the posterior capsule tears or becomes cloudy over a period of time and is cut with a laser to provide a capsulotomy in the posterior capsule. A ruptured capsular rim may be incapable of assuming the taught trampoline-like condition of an intact capsule or rim. As a consequence, a ruptured capsular rim may be incapable of effecting full posterior deflection of a plate-type haptic lens to a distance viewing position against the posterior capsule during and after fibrosis. A ruptured capsule or rim may also permit anterior deflection of the lens during fibrosis. In either case, since the power of intraocular lens is selected for each individual patient and may be dependent upon their spectacle power, and since good vision without glasses requires the lens optic to be situated at precisely the correct distance from the retina throughout the range of accommodation, a simple plate-type haptic lens of the present invention may not be acceptable for use with a ruptured anterior capsule remnant or rim.

FIGS. 10A and 10B illustrate a modified accommodating intraocular lens 400 according to the present invention having a lens body 405 and a pair of haptics 410. As with previous embodiments, haptics 410 have a plate-like shape, and include a reinforced portion 415 and a relatively flexible portion 420. In the embodiment shown, the distal end of haptics 410 includes at least one cut-out portion 425, although other embodiments may not include cut-outs 425. A fixation element 430 is anchored within reinforced portion 415 of haptic 410. In one embodiment, fixation element 430 has a proximal, or anchor, end 435 that is mounted within the thickness of haptic 410 and a distal, substantially T-shaped end, having a pair of arms 440. Arms 440 may be flexible or relative inflexible. The shape of fixation element 430 provides for growth of fibrotic tissue around the distal ends of fixation element 430 to enhance fixation and positioning of the lens 400 in the capsular bag. Fixation elements may be formed from bio-compatible materials such as nylon, polypropylene, polyethylene, polycarbonate or other materials known to those skilled in the art, provided the materials are bio-compatible and have the required physical characteristics, such as flexibility, strength, and the ability to be sterilized.

The fixation elements 430 and haptics 410 are inter-engaged in such a way that the elements 430 and haptics 410 are capable of relative movement lengthwise of the haptics when the haptics flex during accommodation of the lens. Fixation element is typically mounted within the thickness of the haptic 410 during manufacturing of the lens 400. One technique includes insert molding of lens 400 wherein a mold designed to accept fixation element 430 is used to incorporated fixation element 430 into the haptic 410 of lens 400. Alternatively, where lens body 402 is formed from a sufficiently flexible material, a cavity may be formed in the distal ends of haptics 410 sized to receive and retain the anchor portion 435 of fixation element 430. Fixation element 430 may then be inserted into the cavity after the lens body 402 is formed. Mounting fixation element 430 within haptic 410 in this manner necessarily requires the reinforced portion 415 of haptic 410, at least in an area adjacent the cavity, to be sufficiently flexible to allow insertion and retention of the anchor portion 435 of fixation element 430. Alternatively, where the reinforced portion 415 of haptic 410 is relatively inflexible, a fixation element 430 having a shape configured to be inserted into the cavity without stretching reinforced portion 415 in the area of the cavity may be inserted into the cavity and held in place with a suitable adhesive or other means, such as a pin or suture extending through the thickness of the haptic 410 and fixation element 430.

Another embodiment of the accommodating intraocular lens of the present invention including a different embodiment of fixation elements integrally attached to the body of an intraocular lens 450 is depicted in FIGS. 11A and 11B. In this embodiment, lens 450 has a lens body 452 and a pair of haptics 460. As with previous embodiments, haptics 460 have a plate-like shape, and include a reinforced, relatively inflexible, portion 465 and a relatively flexible portion 470. In the embodiment shown, the distal end of haptics 460 includes at least one cut-out portion 475, forming a pair of tabs 480 at the distal end of haptic 460, although other embodiments may not include cut-outs 475 or tabs 480. A fixation element 490 is anchored within reinforced portion 465 of haptic 460. In one embodiment, fixation element 490 is loop shaped and has a proximal, or anchor end 495 that is mounted within the thickness of haptic 460 and a distal end 500. Fixation element 490 may be flexible or relative inflexible. Where fixation element 490 is relatively flexible, the flexibility allows distal end 500 to move proximally towards the optic portion 455 or distally away from optic portion 455 to accommodate capsular bags having varying shapes and diameters. Additionally, the shape of fixation element 490 provides for growth of fibrotic tissue around the distal end of fixation element 430 to enhance fixation and positioning of the lens 400 in the capsular bag. In an alternative embodiment, the distal end 500 of the fixation element 490 may include a hole 505. Although FIG. 11A shows an embodiment of the present invention wherein both fixation elements 490 have holes 505 formed in their distal ends 500, it will be understood that only one of the pair of fixation elements 490 may have a hole 505 formed in its distal end 500.

As in previous embodiments, hole 505 allows for fibrotic tissue to grow through hole 505 to firmly anchor the distal end 500 of fixation element 490 in the capsular bag. Additionally, hole 505 in distal end 500 of fixation element 490 allows a suture to be passed through the hole 505 and tied to retain the fixation elements 490 and lens body 452 in an assembled relation during implantation of the lens 450 in the capsular bag. As described previously, this suture may be removed at the conclusion of surgery, thus releasing the distal ends 500 of fixation elements 490 to spring into the sulcus 40 of the capsular bag 15 (FIG. 1), thus ensuring centration of the lens 450 within the capsular bag 15.

While several specific embodiments of the invention have been illustrated and described, it will be apparent that various modifications can be made without departing from the spirit and scope of the invention. Accordingly, it is not intended that the invention be limited, except as by the appended claims.

I claim:

1. An accommodating intraocular lens comprising:
an optic adapted to focus light toward a retina of an eye;
a pair of generally rectilinear plate haptics joined to and extending from opposite sides of the optic, each of the pair of generally rectilinear plate haptics having a flexible portion and a relatively inflexible portion.

2. The accommodating intraocular lens of claim 1, wherein the relatively inflexible portion includes a material having a flexibility less than the flexibility of the flexible portion for providing increased stiffness disposed within a thickness of the relatively inflexible portion of the haptics.

3. The accommodating intraocular lens of claim 1, wherein the pair of generally rectilinear plate haptics have a length, a width and a thickness and the thickness of the plate haptics is substantially equal along the length and width of the haptics.

4. The accommodating intraocular lens of claim of 3, wherein the relatively inflexible portion includes a material for providing increased stiffness disposed within the thickness of the relatively inflexible portion.

5. The accommodating intraocular lens of claim 1, wherein each of the pair of plate haptics has a proximal end joined to the optic and a distal end and at least one of the pair of plate haptics has at least one opening extending through the plate haptic adjacent the distal end of the at least one plate haptic.

6. The accommodating intraocular lens of claim 1, wherein each of the pair of plate haptics has a proximal end joined to the optic and a distal end and further comprising a fixation element having a distal portion and a proximal end attached to the distal end of at least one of the pair of plate haptics.

7. The accommodating intraocular lens of claim 3, wherein the optic has a width and the width of at least one of the pair of plate haptics along the length of the at least one of the pair of plate haptics is substantially equal to the width of the optic.

8. The accommodating intraocular lens of claim 3, wherein each of the pair of plate haptics includes a proximal end joined with the optic and a distal end and the optic has a width, and wherein the width of the distal end of at least one of the pair of plate haptics is different from the width of the optic.

9. The accommodating intraocular lens of claim 8, wherein the width of the distal end of at least one of the pair of plate haptics is less than the width of the optic.

10. The accommodating intraocular lens of claim 8, wherein the width of the distal end of at least one of the pair of plate haptics is greater than the width of the optic.

11. The accommodating intraocular lens of claim 2, wherein the material is in the form of a mesh.

12. The accommodating intraocular lens of claim 4, wherein the material is in the form of a mesh.

13. The accommodating intraocular lens of claim 6, wherein the fixation element is a relatively flexible loop and wherein the distal portion of the fixation element is capable of flexing from a first position to a second position.

14. The accommodating intraocular lens of claim 6, wherein the fixation element includes a hole extending through the fixation element.

15. The accommodating intraocular lens of claim 14, wherein the hole is located adjacent a distal end of the distal portion of the fixation element.

16. An intraocular lens comprising:
an optic adapted to focus light toward a retina of an eye;
a pair of generally rectilinear plate haptics joined to and extending from opposite sides of the optic, the generally rectilinear plate haptics having a length dimension and a width dimension, the generally rectilinear plate haptics having a substantially equal thickness along the length dimension, the generally rectilinear plate haptics also having a flexible portion and a relatively inflexible portion, the relatively inflexible portion including a relatively non-flexible material having a flexibility less than the flexibility of the flexible portion disposed within the thickness of the relatively inflexible portion of the generally rectilinear plate haptics.

17. The intraocular lens of claim 16, wherein the relatively non-flexible material is formed as a mesh.

18. The intraocular lens of claim 16, wherein the plate haptics have a proximal end joined to the optic and a distal end having a width dimension, and wherein the width dimension of the distal end of the plate haptic is substantially the same as the width dimension of the optic.

19. The intraocular lens of claim 16, wherein the plate haptics have a proximal end joined to the optic and a distal end having a width dimension, and wherein the width dimension of the distal end of the plate haptic is different from the width dimension of the optic.

20. The intraocular lens of claim 19, wherein the width dimension of the distal end of the plate haptic is less than the width dimension of the optic.

21. The intraocular lens of claim 19, wherein the width dimension of the distal end of the plate haptic is greater than the width dimension of the optic.

22. The intraocular lens of claim 16, wherein each of the pair of plate haptics has a proximal end joined to the optic and a distal end, and further comprising a fixation element having a distal portion and a proximal end disposed within the distal end of at least one of the pair of plate haptics.

23. The intraocular lens of claim 22, wherein the fixation element is a relatively flexible loop and wherein the distal portion of the fixation element is capable of flexing from a first position to a second position.

24. The intraocular lens of claim of claim 16, wherein each of the pair of plate haptics is joined to the optic in an angulated fashion.

25. An intraocular lens comprising:
an optic adapted to focus light toward a retina of an eye;
a pair of plate haptics joined to and extending from opposite sides of the optic, the pair of plate haptics having a length dimension and a width dimension, at least one of the pair of plate haptics having a substantially equal thickness along the length dimension, the at least one plate haptic also having a flexible portion and a relatively inflexible portion.

26. The intraocular lens of claim 25, wherein the relatively inflexible portion includes a material for providing increased stiffness disposed within a thickness of the relatively inflexible portion.

27. The intraocular lens of claim 26, wherein the relatively inflexible material is formed as a mesh.

28. The intraocular lens of claim 25, wherein the plate haptics have a proximal end joined to the optic and a distal end having a width dimension, and wherein the width dimension of the distal end of the plate haptic is substantially the same as the width dimension of the optic.

29. The intraocular lens of claim 25, wherein the plate haptics have a proximal end joined to the optic and a distal end having a width dimension, and wherein the width dimension of the distal end of the plate haptic is different from the width dimension of the optic.

30. The intraocular lens of claim 29, wherein the width dimension of the distal end of the plate haptic is less than the width dimension of the optic.

31. The intraocular lens of claim 29, wherein the width dimension of the distal end of the plate haptic is greater than the width dimension of the optic.

32. The intraocular lens of claim 25, wherein each of the pair of plate haptics has a proximal end joined to the optic and a distal end, and further comprising a fixation element having a distal portion and a proximal end disposed within the distal end of at least one of the pair of plate haptics.

33. The intraocular lens of claim 32, wherein the fixation element is a relatively flexible loop and wherein the distal portion of the fixation element is capable of flexing from a first position to a second position.

34. The intraocular lens of claim of claim 25, wherein each of the pair of plate haptics is joined to the optic in an angulated fashion.

35. An intraocular lens comprising:
an optic adapted to focus light toward a retina of an eye;
a pair of generally rectilinear plate haptics joined to and extending from opposite sides of the optic, the generally rectilinear plate haptics each having a flexible portion and a relatively inflexible portion, the relatively inflexible portion having a means for stiffening incorporated withing a thickness of the relatively inflexible portion.

36. The intraocular lens of claim 35, wherein each of the pair of plate haptics has a proximal end joined to the optic and a distal end, and further comprising a fixation element having a distal portion and a proximal end disposed within the distal end of at least one of the pair of plate haptics.

37. The intraocular lens of claim 36, wherein the fixation element is a relatively flexible loop and wherein the distal portion of the fixation element is capable of flexing from a first position to a second position.

38. In an accommodating intraocular lens for implantation in the capsular bag of an eye along the visual axis of the eye, the accommodating intraocular lens having an optic and a pair of plate haptics, the plate haptics configured to provide for movement of the optic along the axis of the eye in response to forces applied to the lens by a ciliary muscle of the eye attached to the capsular bag by zonules to focus light passing through the lens toward a retina of the eye, the improvement comprising:
each of the pair of plate haptics have a proximal end joined to the optic and a distal end, each of the pair of plate haptics also having a thickness, the thickness being substantially equal from the proximal to the distal ends, each of the pair of plate haptics also having a relatively flexible portion located adjacent the proximal end and a relatively inflexible portion having less flexibility than the flexible portion of the haptic located adjacent the distal end, the relatively inflexible portion including a material for providing stiffness to the relatively inflexible portion disposed within the thickness of each of the pair of plate haptics.

39. The intraocular lens of claim 38, wherein each of the pair of plate haptics has a proximal end joined to the optic and a distal end, and further comprising a fixation element having a distal portion and a proximal end disposed within the distal end of at least one of the pair of plate haptics.

40. The intraocular lens of claim 39, wherein the fixation element is a relatively flexible loop and wherein the distal portion of the fixation element is capable of flexing from a first position to a second position.
